(19) United States
(12) Patent Application Publication
(10) Pub. No.: US 2022/0395213 A1
(43) Pub. Date: Dec. 15, 2022

(54) METHODS AND APPARATUS FOR DETERMINING LIKELY OUTCOMES OF AN ELECTROPHYSIOLOGY PROCEDURE

(71) Applicant: CATHETER PRECISION, INC., Mt. Olive, NJ (US)

(72) Inventors: David JENKINS, Flanders, NJ (US); Missiaen HUCK, Flanders, NJ (US)

(21) Appl. No.: 17/346,074

(22) Filed: Jun. 11, 2021

(51) Int. Cl. A61B 5/364; G16H 50/50; G16H 50/70; G16H 10/60; G16H 20/30; G16H 30/20; G16H 30/40; G16H 50/20; G16H 40/67; G06N 20/00; G06K 9/62; G06T 19/20

(52) U.S. Cl. CPC A61B 5/364; G16H 50/50; G16H 50/70; G16H 10/60; G16H 20/30; G16H 30/20; G16H 30/40; G16H 50/20; G16H 40/67; G06N 20/00; G06K 9/6289; G06T 19/20; G06K 2209/051; G06T 2210/41; G06T 2219/2016

(57) ABSTRACT

Various embodiments include methods and diagnostic systems implementing the methods for determining a prognostic prediction of a likelihood of success or a likelihood of complications of an electrophysiology procedure at the identified area of electrophysiological interest. Various embodiments may include generating a patient-specific three-dimensional (3D) cardiac activation and arrythmia localization model identifying an area of electrophysiological interest for performing an electrophysiology procedure to treat the arrythmia, using the 3D heart model to identify heart structures near the identified area of electrophysiological interest, determining a prognostic indication of an electrophysiology procedure performed at the identified area of electrophysiological interest based at least in part on one or more heart structures near the area of electrophysiological interest, and generating an output providing a prognostic indication of an electrophysiology procedure at the identified area of electrophysiological interest based at least in part on the determined likelihood of success.

### BACKGROUND

Cardiac arrhythmia is a group of conditions in which the heartbeat is irregular. There are four main groups of arrhythmia: extra beats, supraventricular tachycardia, ventricular arrhythmia and bradyarrhythmia. As a result of arrhythmia, the heart may not pump efficiently, and can lead to blood clots and heart failure. Cardiac arrhythmia can have a variety of causes, including age, heart (muscle) damage, medications and genetics.

Premature Ventricular Contractions (PVCs) are abnormal or aberrant heart beats that start somewhere in the heart ventricles rather than in the upper chambers of the heart as with normal sinus beats. PVCs typically result in a lower cardiac output as the ventricles contract before they have had a chance to completely fill with blood. PVCs may also trigger Ventricular Tachycardia (VT or V-Tach).

Ventricular tachycardia (VT or V-Tach) is another heart arrhythmia disorder caused by abnormal electrical signals in the heart ventricles. In VT, the abnormal electrical signals cause the heart to beat faster than normal, usually more than 100 beats per minute, with the beats starting in the heart ventricles. VT generally occurs in people with underlying heart abnormalities. VT can sometimes occur in structurally normal hearts, and in such patients the origin of abnormal electrical signals can be in multiple locations in the heart. One common location is in the right ventricular outflow tract (RVOT), which is the route the blood flows from the right ventricle to the lungs. In patients who have had a heart attack, scarring from the heart attack can create a milieu of intact heart muscle and a scar that predisposes patients to VT.

Other common causes for cardiac arrhythmia include defects in the left and/or right ventricle fast activation fibers, the His-Purkinje system, or scar tissue. As a result, the left and right ventricles may not be synchronized. This is referred to as Left Bundle Branch Block (LBBB) or Right Bundle Branch Block (RBBB).

Electrophysiology procedures, such as catheter ablation are common treatments for patients with certain types of cardiac arrhythmia, such as VT and/or symptomatic PVCs. However, some electrophysiology procedures do not always prove effective in resolving arrythmia.

### SUMMARY

Various embodiments provide methods performed by a diagnostic apparatus for determining a likelihood of outcome of a cardiac electrophysiology procedure for treating an arrythmia in a patient. Various embodiments may include using a patient-specific three-dimensional (3D) cardiac activation and arrythmia localization model to identify electrophysiological area of electrophysiological interests of interest for performing an electrophysiology procedure to treat the arrythmia, using the 3D heart model to identify heart structures near the identified area of electrophysiological interest, determining a prognostic indication of an electrophysiology procedure performed at the identified area of electrophysiological interest based at least in part on one or more heart structures near the area of electrophysiological interest, and generating an output providing a prognostic indication of an electrophysiology procedure at the identified area of electrophysiological interest based at least in part on the determined likelihood of success. In some embodiments, the area of electrophysiological interest may include an area of earliest activation within the heart. In some embodiments, the area of electrophysiological interest may include an area of latest activation within the heart. In some embodiments, the area of electrophysiological interest may include an area within the heart between the earliest activation and the latest activation. In some embodiments, the electrophysiology procedure may be or include an ablation procedure or a pacing procedure.

Some embodiments may further include generating the patient-specific 3D cardiac activation and arrythmia localization model by generating a cardiac activation map comprising a 3D heart model that shows propagation of electrical signals through the 3D heart model based on patient electrocardiogram (ECG) data recording during arrythmia events and a 3D heart model that includes structures of the heart, selecting a 3D reference model of the heart and adjusting the 3D reference model based on patient Digital Imaging and Communications in Medicine (DICOM) image data, and generating a 3D mesh reference model based on the patient's DICOM image data, obtaining a 3D image of the patient's torso, and merging the 3D image of the patient's torso with the 3D patient specific heart model to form a patient-specific arrythmia localization and cardiac activation model.

In some embodiments, determining the prognostic indication of the electrophysiology procedure may include determining at least one of a likelihood of success or a likelihood of complications of the electrophysiology procedure at the identified area of electrophysiological interest. In some embodiments, determining at least one of a likelihood of success or a likelihood of complications may include applying the one or more heart structures near the area of electrophysiological interest as model inputs to a predictive model, and obtaining an output from the diagnostic predictive model. Some embodiments may further include applying characteristics of the arrythmia to the diagnostic predictive model.

In some embodiments, the diagnostic predictive model may be maintained in a remote server, and applying the one or more heart structures near the area of electrophysiological interest as model inputs to the diagnostic predictive model may include uploading the one or more heart structures near the area of electrophysiological interest to the remote server, and obtaining an output from the diagnostic predictive model may include receiving the output from the remote server. In some embodiments, the diagnostic predictive model may be stored in memory of the diagnostic apparatus. Some embodiments may further include downloading the diagnostic predictive model or an update to the diagnostic predictive model to memory from a remote server.

Some embodiments may further include uploading to a remote server information regarding characteristics of the arrythmia, information regarding the one or more heart structures near the area of electrophysiological interest, and an indication of one or both of an assessment of success or a summary of complications of a performed electrophysiology procedure performed at the identified area of electrophysiological interest.

Further embodiments include a diagnostic apparatus including one or more processors configured to perform operations of any of the methods summarized above. Further embodiments include a non-transitory processor-readable medium having stored thereon processor-executable instructions configured to cause a processor of a diagnostic apparatus to perform operations of any of the methods summarized above.

Further embodiments include methods that may be performed by a server or a similar computing device for developing and/or refining a predictive model for determining a likelihood of outcome of a cardiac electrophysiology procedure for treating an arrythmia in a patient. Such embodiments may include receiving from diagnostic systems information regarding characteristics of an arrythmia of a patient, one or more heart structures near an area of electrophysiological interest on the patient's heart, and an indication of one or both of an assessment of success or a summary of complications of an electrophysiology procedure performed on the patient's heart at the identified area of electrophysiological interest, using the received information to generator or update a diagnostic model that is configured to output a prognostic indication of an electrophysiology procedure performed at the identified area of electrophysiological interest based at least in part on one or more heart structures near the area of electrophysiological interest, and providing the diagnostic predictive model to diagnostic systems.

In some embodiments, the diagnostic model may be configured to output the prognostic indication of an electrophysiology procedure including at least one of a likelihood of success or a likelihood of complications of an electrophysiology procedure at the identified area of electrophysiological interest based at least in part on one or more heart structures near the area of electrophysiological interest.

In some embodiments, providing the diagnostic predictive model to diagnostic systems may include downloading the diagnostic predictive model to diagnostic systems for storage in memory of the diagnostic systems.

In some embodiments, providing the diagnostic predictive model to diagnostic systems may include receiving, from a diagnostic system, information regarding a potential electrophysiology procedure including information regarding at least one or more heart structures near an area of electrophysiological interest, and determining at least one of a likelihood of success or a likelihood of complications of an electrophysiology procedure at the area of electrophysiological interest based at least in part on the one or more heart structures near the area of electrophysiological interest, and communicating the determined likelihood of success or likelihood of complications of the potential electrophysiology procedure to the diagnostic system.

In some embodiments, using the received information to generator or update a diagnostic model that is configured to output a prognostic indication of an electrophysiology procedure performed at the identified area of electrophysiological interest based at least in part on one or more heart structures near the area of electrophysiological interest may include using machine learning technology to generate or update the diagnostic model based on information regarding characteristics of the arrythmia, the one or more heart structures near the area of electrophysiological interest, and an indication of one or both of an assessment of success or a summary of complications of a performed electrophysiology procedure performed at the identified area of electrophysiological interest received from a plurality of diagnostic systems.

Further embodiments include a server or other computing system configured to perform operations of any of the server methods summarized above. Further embodiments include a non-transitory processor-readable medium having stored thereon processor-executable instructions configured to cause a server or other computing system to perform operations of any of the server methods summarized above.

### BRIEF DESCRIPTION OF THE DRAWINGS

The accompanying drawings, which are incorporated herein and constitute part of this specification, illustrate example embodiments of the invention, and together with the general description given above and the detailed description given below, serve to explain the features of the invention.

FIG. 1 is an example of a 3D model of a heart according to various embodiments.

FIG. 2 is a plan view of a 3D model of electrical activation of a heart according to various embodiments.

FIG. 3 is a schematic representation of a process for determining a diagnostic prediction of an electrophysiology procedure according to various embodiments.

FIG. 4A is a system block diagram of a diagnostic system configured to determine a diagnostic prediction of an electrophysiology procedure according to various embodiments.

FIG. 4B is a system block diagram of a server system configured to generate a diagnostic prediction model according to various embodiments.

FIG. 5A is a process flow diagram illustrating an example method for determining a diagnostic prediction of an electrophysiology procedure according to various embodiments.

FIG. 5B is a process flow diagram illustrating a further example method that may be implemented as part of determining a diagnostic prediction of an electrophysiology procedure according to some embodiments.

FIG. 6 is a process flow diagram illustrating further operations that may be included as part of a method for determining a diagnostic prediction of an electrophysiology procedure according to some embodiments.

FIG. 7A is a process flow diagram illustrating operations that may be performed by a remote computing system for generating and updating diagnostic predictive models used for determining a diagnostic prediction of an electrophysiology procedure according to some embodiments.

FIG. 7B is a process flow diagram illustrating further operations that may be included as part of a method for generating and updating diagnostic predictive models used for determining a diagnostic prediction of an electrophysiology procedure.

FIG. 8 is a component block diagram illustrating an example mobile computing device suitable for use with the various embodiments.

FIG. 9 is a component block diagram illustrating an example server suitable for use with the various embodiments.

### DETAILED DESCRIPTION

The various embodiments will be described in detail with reference to the accompanying drawings. Wherever possible, the same reference numbers will be used throughout the drawings to refer to the same or like parts. References made to particular examples and implementations are for illustrative purposes, and are not intended to limit the scope of the invention or the claims.

Various embodiments include methods that may performed by a diagnostic apparatus for determining a likelihood of outcome of a cardiac electrophysiology procedure for treating a cardiac condition, such as an arrythmia in a patient. Embodiment methods may be performed by a processor of a diagnostic apparatus or system. By the diagnostic apparatus determining a likelihood of success or complications for an indicated electrophysiology procedures, physicians can make better decisions regarding whether such an electrophysiology procedure should be conducted or whether other treatment options should be considered.

Various embodiment include a diagnostic apparatus, which may be a work station, a laptop computer, or a dedicated computing system that is configured with processor-executable instructions to use a patient-specific 3D cardiac activation and arrythmia localization model to identify an area of electrophysiological interest for performing an electrophysiology procedure to treat the arrythmia. The diagnostic apparatus may use a patient-specific 3D heart model (which may be the patient-specific 3D cardiac activation and arrythmia localization model) to identify heart structures near the identified area of electrophysiological interest, and automatically determining a prognostic indication of an electrophysiology procedure performed at the identified area of electrophysiological interest based at least in part on one or more heart structures near the area of electrophysiological interest. The diagnostic apparatus may be configured to generate an output for review by a physician, including providing a prognostic indication of performing an electrophysiology procedure at the identified area of electrophysiological interest based at least in part on the determined likelihood of success.

In some embodiments, the patient-specific 3D cardiac activation and arrythmia localization model used to identify the area of electrophysiological interest for performing an electrophysiology procedure to treat the arrythmia may be created by the diagnostic apparatus by generating a cardiac activation map including a 3D heart model that shows propagation of electrical signals through the 3D heart model based on patient electrocardiogram (ECG) data recording during arrythmia events and a 3D heart model. The diagnostic apparatus and/or a physician may use the patient-specific arrythmia localization and cardiac activation model to identify an area of electrophysiological interest for performing an electrophysiology procedure to treat the arrythmia. In various embodiments, the area of electrophysiological interest may include an area of earliest activation within the heart, an area of latest activation within the heart, and/or an area within the heart between the earliest activation and the latest activation. The electrophysiology procedure may be or include an ablation procedure or a pacing procedure.

To generate a 3D heart model, a 3D reference model of the heart that includes structures of the heart (e.g., ventricles, valves, blood vessels, wall thickness, etc.) may be selected by the diagnostic apparatus, such as from a library of models stored in memory. The selection of an appropriate reference 3D heart model may be accomplished by comparing patient Digital Imaging and Communications in Medicine (DICOM) image data to a number of different reference models to find one that best batches the patient. This selection may be performed automatically by the diagnostic apparatus. The diagnostic apparatus may then adjust the selected 3D reference model of the heart based on the patient DICOM image data. In this operation, locations and orientations of modeled structures of the heart may be adjusted consistent with actual locations and orientations of such tissue structures as determined or imaged in the patient's DICOM data. In other words, the selected the 3D reference model of the heart may be adjusted to conform to the locations, sizes and orientations of heart structures revealed in DICOM images.

To further orient the 3D heart model in the patient, a 3D image of the patient's torso may be taken using a 3D camera, such as before, during or after recording ECG data. The 3D camera may be part of the diagnostic apparatus, a separate apparatus that is coupled to the diagnostic apparatus by wired or wireless communication link, or a separate apparatus that stores the image data to a medium (e.g., local memory or portable memory) that can be connected to the diagnostic apparatus (e.g., physically connected or connected via a wired or wireless network connection).

The diagnostic apparatus may receive the 3D image data and then merge the cardiac activation map, the adjusted 3D reference model of the heart, and the 3D image of the patient's torso to form a patient-specific arrythmia localization and cardiac activation model that includes internal structures of the heart. In this process, ECG electrodes and/or fiducial markers on the patient's torso when the 3D image is obtained may be used by the diagnostic apparatus as reference points for aligning the different imagery during the process of merging the heart models with DICOM imagery to generate a patient-specific 3D heart model, such as the patient-specific 3D arrythmia localization and cardiac activation model.

Finally, the diagnostic apparatus may use a patient-specific 3D heart model (which may be the patient-specific 3D cardiac activation and arrythmia localization model) to identify heart structures that are located at or near the identified area of electrophysiological interest, and use that information as inputs to a predictive model to determine an assessment or prognosis (referred to herein as a “prognostic indication”) for performing and electrophysiology procedure at the identified area of electrophysiological interest. In some embodiments, determining the prognostic indication of the electrophysiology procedure may include determining at least one of a likelihood of success and/or a likelihood of complications of the electrophysiology procedure at the identified area of electrophysiological interest. For example, the diagnostic apparatus may output an indication of the likelihood that a pacing treatment (e.g., attaching a pacemaker lead) at the identified area of electrophysiological interest will result in successful pacing of the patient's heart to resolve the underlying medical condition. As another example, the diagnostic apparatus may output an indication of the likelihood that an ablation performed at the identified area of electrophysiological interest will result in successful in treatment of the patient's arrythmia condition and/or complications.

The diagnostic predictive model used by the diagnostic apparatus to determine the prognostic indication may be generated based upon the collective results of numerous electrophysiology procedures performed at various locations within patient hearts, which may be gathered and analyzed by a central computing system, such as a remote server. For example, information regarding complete electrophysiology procedures, particularly locations where the electrophysiology procedure was performed with respect to heart structures, and reports of the degree of success and/or complications resulting from each electrophysiology procedure may be aggregated by a server or similar computing device, and then analyzed to generate the diagnostic predictive model by correlating successes and/or complications of electrophysiology procedures to the location on the heart and/or heart structures at or near the area of electrophysiological interest. In some embodiments, the diagnostic predictive model may be generated using machine learning methods with the accumulated information regarding electrophysiology procedures serving as a training database. Once the diagnostic predictive model is generated, the remote server may provide the model to diagnostic apparatus for use in the various embodiments described herein. Further, diagnostic apparatuses may be configured to upload information regarding completed electrophysiology procedures and their associated indications of success and/or complications for use by the server in improving the diagnostic predictive model over time.

In some embodiments, determining at least one of a likelihood of success or a likelihood of complications may include using information regarding the one or more heart structures at or near the area of electrophysiological interest as model inputs to the diagnostic predictive model, and obtaining an output from the diagnostic predictive model. In some embodiments, inputs to the diagnostic predictive model may further include characteristics of the arrythmia in addition to the one or more heart structures at or near the area of electrophysiological interest. In some embodiments, the diagnostic predictive model may be stored in memory of the diagnostic apparatus, such as after downloading the diagnostic predictive model or an update to the diagnostic predictive model from a remote server to local memory.

In some embodiments, the diagnostic predictive model may be maintained in a remote server instead of a memory of the diagnostic apparatus, and the diagnostic apparatus may apply the one or more heart structures at or near the area of electrophysiological interest as model inputs to the diagnostic predictive model by uploading information regarding the one or more heart structures at near the area of electrophysiological interest to the remote server, and receiving an output from the diagnostic predictive model may include receiving the output from the remote server.

In some embodiments, the diagnostic apparatus may provide information to support development and refinement of the diagnostic predictive model by uploading to the remote server information regarding performed electrophysiology procedures, such as information regarding the one or more heart structures at or near the area of electrophysiological interest, characteristics of the arrythmia, and an indication of one or both of an assessment of success or a summary of complications of each electrophysiology procedure performed.

Various embodiments also include methods that may be performed by a remote server or similar computing system for developing and refining the diagnostic predictive model for predicting likely outcomes of particular electrophysiology procedures based on information received from physicians and diagnostic system. Such embodiments may include the server or similar computing system receiving from diagnostic systems information regarding characteristics of an arrythmia of a patient, one or more heart structures near an area of electrophysiological interest on the patient's heart, and an indication of one or both of an assessment of success or a summary of complications of an electrophysiology procedure performed on the patient's heart at the identified area of electrophysiological interest. Such information may be received via a network, such as the Internet, and stored in memory of the server or computing system.

After receiving such information from multiple diagnostic system, the server or other computing system may use the received information to generate, refine or update a diagnostic predictive model that is configured to output a prognostic indication of an electrophysiology procedure performed at the identified area of electrophysiological interest based at least in part on one or more heart structures near the area of electrophysiological interest. The diagnostic predictive model may be configured to output the prognostic indication of an electrophysiology procedure including at least one of a likelihood of success or a likelihood of complications of an electrophysiology procedure at the identified area of electrophysiological interest based at least in part on one or more heart structures at or near the area of electrophysiological interest. The generation and/or refinement of the diagnostic predictive model may involve various techniques for recognizing patterns within large data sets. For example, machine learning technology may be used to generate or update the diagnostic model by training the model using information from completed electrophysiology procedures including information such as information regarding one or more heart structures at or near the area of electrophysiological interest, characteristics of the arrythmia, and an indication of one or both of an assessment of success or a summary of complications of a performed electrophysiology procedure performed at the identified area of electrophysiological interest received from a plurality of diagnostic systems.

The server or similar computing system may provide the diagnostic predictive model to diagnostic systems for use in the embodiment methods as described above. In some embodiments, this may involve downloading the diagnostic predictive model to diagnostic systems for storage in memory of the diagnostic systems. In other embodiments, this may involve receiving, from a diagnostic system, information regarding a potential electrophysiology procedure including information regarding at least one or more heart structures at or near an area of electrophysiological interest, determining at least one of a likelihood of success or a likelihood of complications of an electrophysiology procedure at the area of electrophysiological interest based at least in part on the one or more heart structures near the area of electrophysiological interest, and communicating the determined likelihood of success or likelihood of complications of the potential electrophysiology procedure to the diagnostic system.

The term electrocardiogram (ECG) is used herein to refer to any method that (preferably non-invasively) correlates actual electrical activity of the heart muscle to measured or derived (electrical activity) of the heart. In case of a classical electrocardiogram, the differences in potential between electrodes on the body surface are correlated to the electrical activity of the heart. Derived ECG's can also be obtained in other ways (e.g. by measurement made by a so-called ICD (Implantable Cardioverter Defibrillator)). In order to obtain such a functional image an estimation of the movement of the electrical activity has to be provided.

During normal conduction, cardiac activation begins within both the left ventricular (LV) and right ventricular (RV) endocardium. In particular, electrical impulses (i.e., depolarization waves) travel substantially simultaneously through both the left and right ventricles. By analyzing electrical signals gathered in an ECG procedure using multiple electrodes on the patient's body, a diagnostic apparatus may develop an activation model of the patient's heart that may reveal conductive pathways and/or locations of interruptions to the depolarization waves. Using the information provided in such an activation model may enable the diagnostic apparatus and/or clinicians to identify a location for performing an electrophysiology procedure in order to treat an arrhythmia condition in a patient.

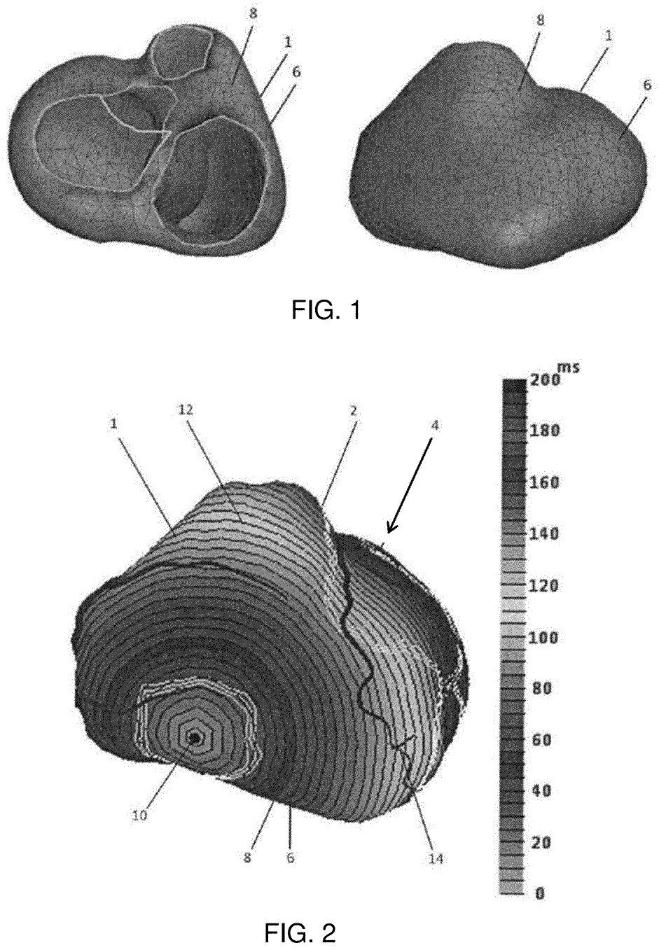

FIG. 1 shows a three-dimensional (3D) activation model of a heart 1 seen in two different directions. The 3D model includes a mesh 6 representing an outer surface of the heart near the myocardial surface. In this example, the activation model also may include the septal wall. The mesh 6 features a plurality of nodes 8. In this example, the mesh is a triangular mesh in which the surface of the heart is approximated by adjoining triangles.

FIG. 2 is an example of an activation model 4 of a heart that may be generated based on ECG measurements made on a patient. In general, a 3D activation model 4 may include a mesh 6 representing a ventricular surface of the heart. FIG. 2 shows an outer surface of the ventricular myocardium with septal wall as represented in FIG. 1. The mesh 6 has a plurality of nodes 8. In the illustrated example, the heart 1 is electrically stimulated at a stimulation location 10. Upon electrical stimulation at the stimulation location 10, the electrical signals will travel through the heart tissue. Hence, different parts of the heart will be activated at different times. Each location on the heart has a particular delay relative to the initial stimulation. Each node 8 has associated therewith a value representative of a time delay between stimulation of the heart 1 at the stimulation location 10 and activation of the heart at that respective node 8. Locations that share the same delay time are connected by isochrones 12 in FIG. 2. In this application, isochrones are defined as lines drawn on a 3D heart surface model connecting points on the model at which the activation occurs or arrives at the same time. The delay time for nodes across the heart surface in this example is also displayed by differing shading. The vertical bar indicates the time delay in milliseconds associated with the respective shading.

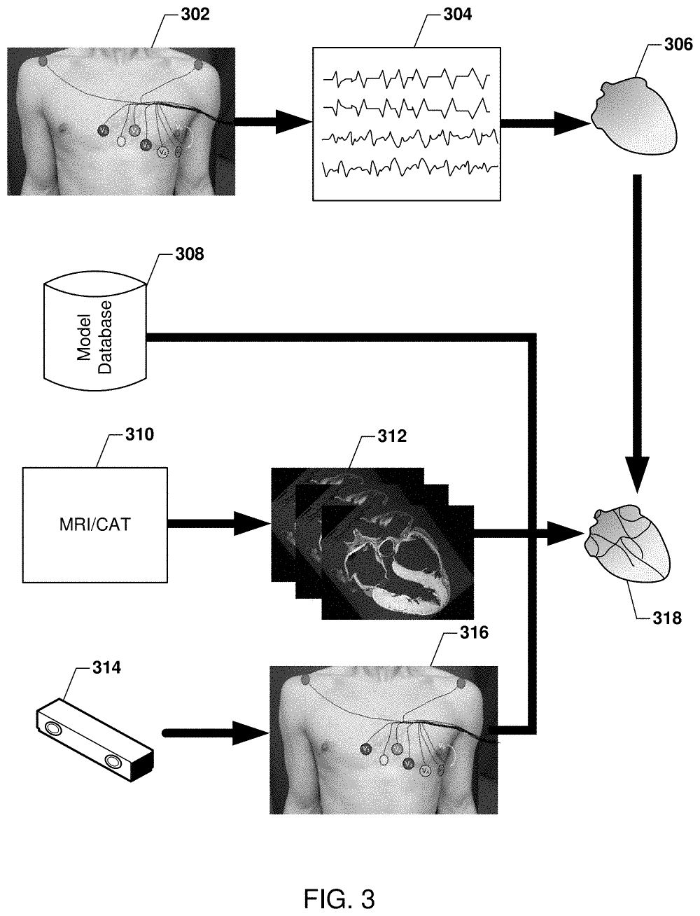

FIG. 3 illustrates the generation of a patient-specific heart model that may be performed by a diagnostic apparatus by combining an activation model of the heart showing conductive pathways and isochrones with a patient specific structural model of the patient's heart.

As described above, electrical activation of the patient's heart may be obtained using a 12-lead ECG procedure 302 that produces cardiac electrical signal information 304 from several electrodes. The cardiac electrical signal information 304 may be stored in memory and analyzed by a computing system—which may be part of the diagnostic apparatus or a separate system—to generate an activation model 306 of the patient's heart. As described with reference to FIG. 2, the activation model 306 may include detail information regarding how depolarization waves transit the heart tissues, which can reveal locations where arrhythmias are initiated and thus locations for performing electrophysiology procedures. The generation of the activation model 306 based on ECG data may use methods and systems that are currently available or may be developed in the future.

A patient-specific heart model that shows structural details of the heart in combination with the activation model may be developed by generating a 3D structural model of the patient's heart that can be combined or merged with the activation model 306 in an electrocardiographic imaging (ECGI) method. A patient-specific 3D anatomical heart model may be generated by using information from magnetic resonance imaging (MRI), computed tomography (CT), positron emission tomography (PET), and/or ultrasound generated DICOM images 312 to modify a 3D anatomical model of the heart and torso obtained from a database 308 including a plurality of 3D anatomical models. A 3D anatomical model of a heart and torso showing closest conformity to the patient's anatomy based on the DICOM images may be selected from the database 308, and then this model adjusted or modified to reflect the actual locations of various anatomical structures in the torso and the heart as shown in the DICOM images 312. This operation of selecting a suitable anatomical model and then adjusting the model to match the patient may be performed automatically by the diagnostic system or another system.

To further match the anatomical model to the patient's heart, a 3D camera 314 may be used to obtain 3D images 316 of the patient's torso during the ECG procedure while the cardiac electrical signal information 304 is recorded. The 3D images of the patient taken during the ECG procedure enables the diagnostic apparatus or another computing system to determine the body locations of fiducial markers and the electrodes used to record the cardiac electrical signal information 304. Again, by capturing the 3D images of the patient's torso during the ECG procedure, the diagnostic apparatus may use the locations in the 3D images of ECG electrodes and/or fiducial markers on the patient's torso as reference points for aligning the different imagery during the process of merging heart and torso models with DICOM imagery to generate the patient-specific arrythmia localization and cardiac activation model. The 3D image and the torso model may be aligned, and the locations of the electrodes in the anatomical model may be adjusted to coincide with the electrode locations in the 3D image. Knowledge of the location of the ECG electrodes relative to the heart, and in particular the V1-6 precordial electrodes, may be especially important for accurately computing the onset location of arrythmias such as PVC.

The 3D electrical activation model 306 may be merged or combined with the patient-specific 3D heart model and 3D images 316 of the patient to generate a patient-specific 3D cardiac activation and arrythmia localization model 318. The patient-specific 3D cardiac activation and arrythmia localization model 318 may include cardiac structure information, such as locations of the four cardiac valves and supporting fibrous tissues, shapes, sizes and locations of the right and left atriums and right and left ventricles, and cardiac blood vessels and veins in the myocardium. The ECGI method may be automated and performed by a diagnostic apparatus or other computing systems to combine data of ECG signals with a patient-specific 3D anatomical model of the heart (as well as other features including the lungs and torso) to compute the positions of the cardiac isochrones relative to heart structures.

The patient-specific 3D cardiac activation and arrythmia localization model 318 may also include information regarding scar tissue. Scar tissue locations may be obtained from delayed enhancement MRI images. Scar tissue can be simulated in the 3D electrical activation model 306 by reducing the propagation velocity of electrical signals through the scar tissue locations. Scar tissue can also be accounted for by slowing the transition from one node to another to very slow or non-transitional for the areas in the heart wall where scar tissue is present. Alternatively, the locations of scar tissue obtained from MRI images may be included in the patient-specific 3D anatomical heart model that is combined with the 3D electrical activation model 306 to yield the patient-specific 3D cardiac activation and arrythmia localization model 318.

The patient-specific 3D cardiac activation and arrythmia localization model 318 may then be used by the diagnostic apparatus to identify one or more locations for conducting ablation therapy to treat arrhythmias, as well as identify the heart structures that are at or near the identified area of electrophysiological interest(s).

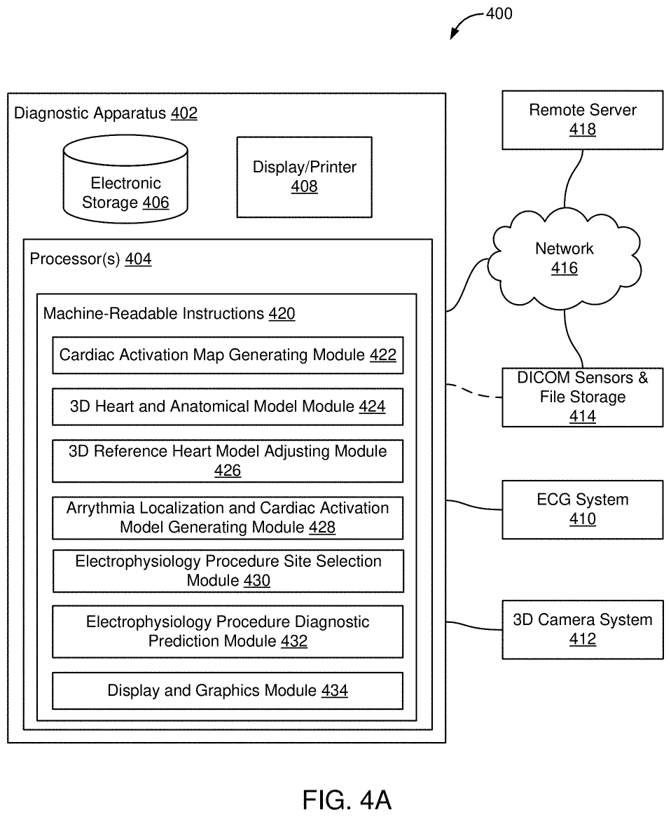

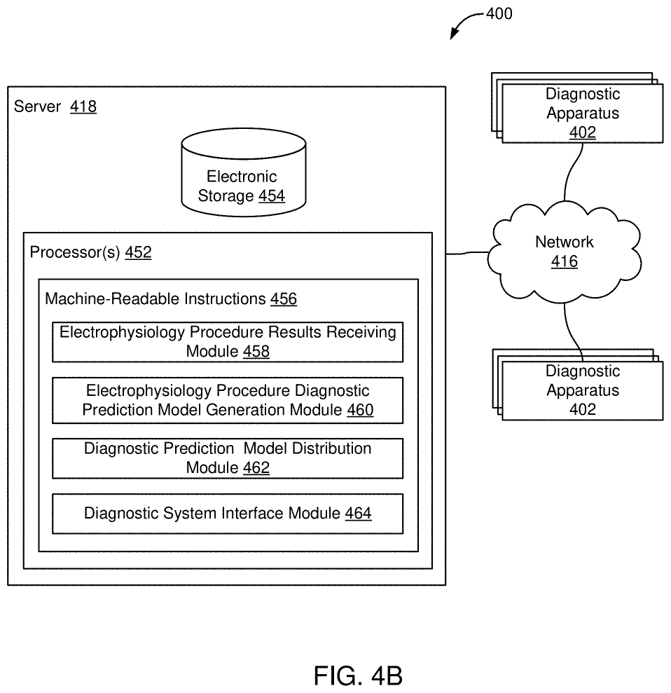

FIG. 4A is a component block diagram illustrating an example system 400 implementing various embodiments for providing a diagnostic indication or metric of success and/or complications from performing an electrophysiology procedure at a given location on a patient's heart. The system 400 may include a diagnostic apparatus 402 that is configured to perform operations of the various embodiment methods. A diagnostic apparatus 402 may include one or more processors 404 that are coupled to memory in the form of electronic storage 406, and to a display, printer and/or other output device 408. The diagnostic apparatus 402 may be coupled to or in some embodiments include an electrocardiogram (ECG) system 410, a 3D camera system 412, and capability for receiving DICOM data files from DICOM sensors and/or file storage 414.

In some embodiments, the ECG system 410 may be part of the diagnostic apparatus 402. In other embodiments, the diagnostic apparatus 402 may be coupled to a separate ECG system 410 via any mechanism that enables ECG data to be received by the processor(s) 404, including but not limited to a data cable between the two devices, a wireless data connection (e.g., via a Bluetooth wireless network connection, a Wi-Fi local area wireless network (WLAN) or wireless wide area network (WWAN) connection to a local network or the Internet, etc.) to the ECG system 410 and/or a memory storing the ECG data, or a portable memory storage device (e.g., a USB memory device) on which the ECG data is stored. As some patients may experience episodic arrythmias, in some embodiments, the ECG data may be recorded using a portable ECG recording device, such as a Holter-type device, which may be connected to the diagnostic apparatus 402 to download recorded ECG data.

In some embodiments, the 3D camera system 412 may be part of the diagnostic apparatus 402. In other embodiments, the diagnostic apparatus 402 may be coupled to a separate 3D camera system 412 via any mechanism that enables image data to be received by the processor(s) 404, including but not limited to a data cable between the two devices, a wireless data connection (e.g., via a Bluetooth wireless network connection, WLAN connection to a local network, etc.) to the 3D camera system 412 and/or a memory storing the image data, or a portable memory storage device (e.g., a USB memory device or flash memory chip) on which the image data is stored.

The diagnostic apparatus 402 may be configured to receive DICOM data files from DICOM sensors (e.g., MRI, CT, x-ray, PET, ultrasound, and other systems) and/or DICOM file storage 414 via any mechanism that enables ECG data to be received by the processor(s) 404, including but not limited to a data cable between two devices, a wireless data connection (e.g., via a Bluetooth wireless network connection, a WLAN or WWAN connection to a local network or the Internet, etc.) to the ECG system 410 and/or a memory storing the ECG data, or a portable memory storage device (e.g., a USB memory device) on which the ECG data is stored.

The diagnostic apparatus 402 may further include a wired or wireless network interface to a network 416 (e.g., a WLAN, a WWAN, and/or the Internet) or connecting to a remote server 418 that is configured to perform some of the operations of various embodiments.

The processor(s) 404 of the diagnostic apparatus 402 may be configured by machine-readable instructions 420, which may include one or more instruction modules. The instruction modules may include computer program modules. The instruction modules may include one or more of a cardiac activation map generating module 422, a 3D heart and anatomical model module 424, a 3D reference heart model adjusting module 426, an arrhythmia localization and cardiac activation model generating module 428, an ablation site selection module 430, an electrophysiology procedure diagnostic prediction module 432, and a display and graphics module 432.

The cardiac activation map generating module 422 may include instructions configured to cause one or more processors 404 to perform operations of using ECG data to generate an activation model of the patient's heart as described herein.

The 3D heart and anatomical model module 424 may include instructions configured to cause one or more processors 404 to perform operations including selecting from memory (e.g., electronic storage 406) or remote data sources (e.g., a remote server 418) a heart and torso reference anatomical model that matches or closely resembles anatomical features of the patient. Such operations may involve using patient imagery (e.g., obtained from the 3D camera system 412 and/or DICOM images) to identify sizes, locations and orientations of various anatomical structures, and using that information to select one reference model from among a number of stored reference anatomical models with anatomical structures like or similar to those of the patient. The operations may further include obtaining or downloading the selected reference anatomical model.

The 3D reference heart model adjusting module 426 may include instructions configured to cause one or more processors 404 to perform operations including adjusting the size, orientation and position of anatomical features (e.g., locations of the four cardiac valves and supporting fibrous tissues, shapes, sizes and locations of the right and left atriums and right and left ventricles, locations of cardiac blood vessels and veins in the myocardium, locations of scar tissue, and other structures) in the selected reference anatomical model so as to more closely match the corresponding anatomical structures of the patient based on the DICOM data. In this manner, a 3D reference model of the patient's heart and surrounding tissues that closely resembles the patient' heart and torso may be generated.

The arrhythmia localization and cardiac activation model generating module 428 may include instructions configured to cause one or more processors 404 to perform operations including merging or combining the 3D cardiac activation model with the patient-specific 3D heart model and 3D images of the patient's torso to generate a patient-specific 3D cardiac activation and arrythmia localization model, such as using methods described with reference to FIG. 3.

The ablation site selection module 430 may include instructions configured to cause one or more processors 404 to perform operations including using the patient-specific 3D cardiac activation and arrythmia localization model to automatically identify a location for performing an ablation to treat an arrhythmia. Such operations may include using the patient-specific 3D cardiac activation and arrythmia localization model to identify sources or initiating sites of an arrhythmia based upon the activation isochrones, and determining the location of such sites on the heart model to identify the area of electrophysiological interest. Such operations may further include identifying heart structures (e.g., locations of the four cardiac valves and supporting fibrous tissues, shapes, sizes and locations of the right and left atriums and right and left ventricles, locations of cardiac blood vessels and veins in the myocardium, locations of scar tissue, and other structures) at or near the identified area of electrophysiological interest.

The electrophysiology procedure diagnostic prediction module 430 may include instructions configured to cause one or more processors 404 to perform operations including using a diagnostic prediction model to determine a prognostic indication of an electrophysiology procedure performed at the identified area of electrophysiological interest based at least in part on one or more heart structures at or near the identified area of electrophysiological interest. These operations may include obtaining the diagnostic prediction model from local memory (e.g., the electronic storage 406) or from a remote server 418, and then using the heart structures at or near the identified area of electrophysiological interest as inputs to the diagnostic prediction model. The diagnostic prediction model may output a likelihood of success or a likelihood of complications of an electrophysiology procedure performed at the identified area of electrophysiological interest. As described herein, the diagnostic prediction model may be developed through analysis of information regarding the characteristics and outcomes of a number of electrophysiology procedures.

The display and graphics module 434 may include instructions configured to cause one or more processors 404 to perform operations including generating graphics including indications of a likelihood of success or a likelihood of complications of an electrophysiology procedure for output via a display unit, a printer, a messaging unit, and/or other output devices.

FIG. 4B is a component block diagram illustrating an example server 418 suitable for implementing various embodiments for generating and maintaining a diagnostic prediction model through analysis of information regarding the characteristics and outcomes of a number of electrophysiology procedures. The example server 418 may be part of the system 400 and may include one or more processors 452 that are coupled to electronic storage 454 and include a network interface for communicating via the network 416 with various diagnostic apparatuses 402.

The processor(s) 452 of the server 418 may be configured by machine-readable instructions 420, which may include one or more instruction modules. The instruction modules may include computer program modules. The instruction modules may include one or more of an electrophysiology procedure results receiving module 458, an electrophysiology procedure diagnostic prediction model generation module 460, and a prediction model distribution module 462.

The electrophysiology procedure results receiving module 458 may include instructions configured to cause a one or more processors 452 to perform operations including receiving from multiple diagnostic apparatuses 402 information regarding the results of electrophysiology procedures to treat the arrhythmia that have been performed on various patients. In particular, the operations may include automatically connecting to diagnostic apparatuses 402 via a network 416, and receiving information regarding the nature of electrophysiology procedures that have been performed (e.g., the nature of the arrhythmia, the location of the ablation and/or heart structures at or near the area of electrophysiological interest) as well as indications of the success and/or complications of the electrophysiology procedures. As part of the operations, information received regarding the results of electrophysiology procedures may be stored in the electronic storage 454, such as in a database suitable for use in analysis performed by the electrophysiology procedure diagnostic prediction model generation module 460.

The electrophysiology procedure diagnostic prediction model generation module 460 may include instructions configured to cause one or more processors 452 to perform operations including using accumulated information regarding the results of electrophysiology procedures to generate a diagnostic prediction model that can output an indication of success and/or complications based at least on a location of the electrophysiology procedure, particularly with respect to nearby heart structures. The operations may include using any known analysis technique for recognizing patterns within a large data set and correlating the recognized patterns into a predictive model. In some embodiments, the operations may include using a machine learning or neural network analysis to generate an artificial intelligence (AI) predictive model. In such embodiments, the operations may include using information regarding the results of several electrophysiology procedures, such as received and stored by the electrophysiology procedure results receiving module 458, as a training database to train a machine learning system, the output of which is the diagnostic prediction model. The operations may further include continuing to refine or update the diagnostic prediction model as further information is received regarding electrophysiology procedures that have been performed to treat the arrhythmia including indications of the success and/or complications of such procedures.

The diagnostic prediction model distribution module 462 may include instructions configured to cause one or more processors 452 to perform operations including interfacing with diagnostic apparatuses 402 to make the diagnostic prediction model available for downloading or remote access. In some embodiments, the operations may include downloading the diagnostic prediction model to various diagnostic apparatuses 402 via a network 416 (e.g., the Internet) for storage in their local memory. Such embodiments may also include periodically downloading updates of the diagnostic prediction model to the various diagnostic apparatuses 402. In some embodiments, the operations may include using the diagnostic prediction model within the server to provide predictions of success and/or complications to diagnostic apparatuses 402 in response to receiving inquiries that include information regarding the location on the heart and/or heart structures at or near an indicated area of electrophysiological interest.

The electronic storage 406, 454 may include non-transitory storage media or memory device that electronically stores information. The electronic storage media of electronic storage 406, 454 may include one or both of system storage that is provided integrally (i.e., substantially non-removable) with the diagnostic apparatus 402 or the server 418 and/or removable storage that is removably connectable to the diagnostic apparatus 402 or the server 418 via, for example, a port (e.g., a universal serial bus (USB) port, a firewire port, etc.) or a drive (e.g., a disk drive, etc.). Electronic storage 406, 454 may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. Electronic storage 406, 454 may include one or more virtual storage resources (e.g., cloud storage, a virtual private network, and/or other virtual storage resources). Electronic storage 406, 454 may store software algorithms, information determined by processor(s) 404, 452, information received from the diagnostic apparatus 402 or the server 418, or other information that enables the diagnostic apparatus 402 or the server 418 to function as described herein.

The processor(s) 404, 452 may include one or more of a digital processor, an analog processor, a digital circuit designed to process information, an analog circuit designed to process information, a state machine, and/or other mechanisms for electronically processing information. Although the processor(s) 404, 452 are illustrated as single entities, this is for illustrative purposes only. In some embodiments, the processor(s) 404,452 may include a plurality of processing units and/or processor cores. The processing units may be physically located within the same device, or processor(s) 404,452 may represent processing functionality of a plurality of devices operating in coordination. The processor(s) 404,452 may be configured to execute modules 420-434 and modules 456-464 and/or other modules by software; hardware; firmware; some combination of software, hardware, and/or firmware; and/or other mechanisms for configuring processing capabilities on processor(s) 404,452. As used herein, the term “module” may refer to any component or set of components that perform the functionality attributed to the module. This may include one or more physical processors during execution of processor-executable instructions by a processor, circuitry, hardware, or any other components.

The description of the functionality provided by the different modules 408-414 and modules 436-446 is for illustrative purposes, and is not intended to be limiting, as any of modules 408-414 and modules 436-446 may provide more or less functionality than is described. For example, one or more of the modules 408-414 and modules 436-446 may be eliminated, and some or all of its functionality may be provided by other modules 408-414 and modules 436-446. As another example, the processor(s) 404,452 may be configured to execute one or more additional modules that may perform some or all of the functionality attributed below to one of the modules 408-414 and modules 436-446.

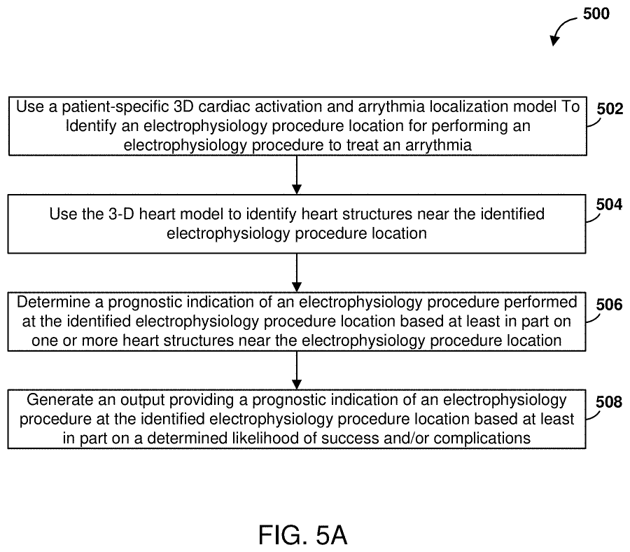

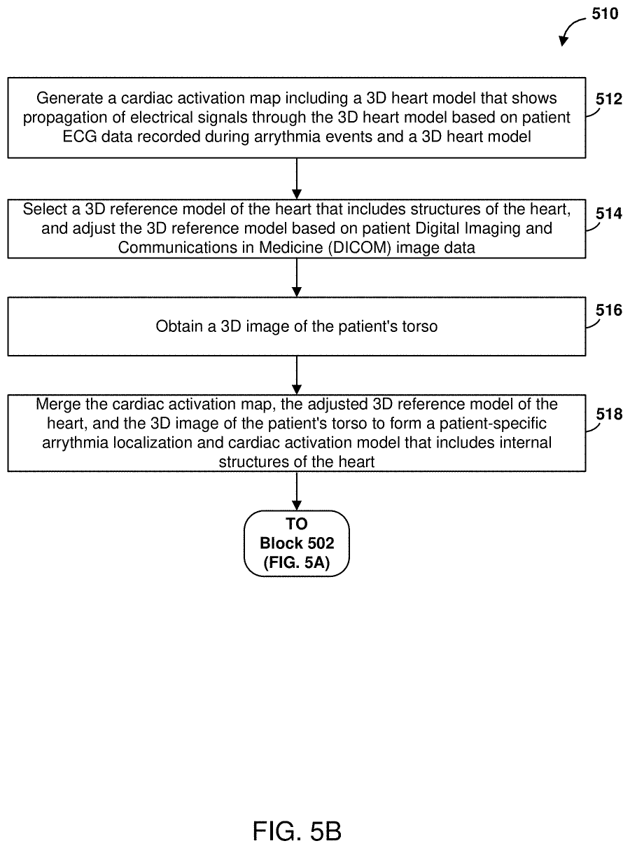

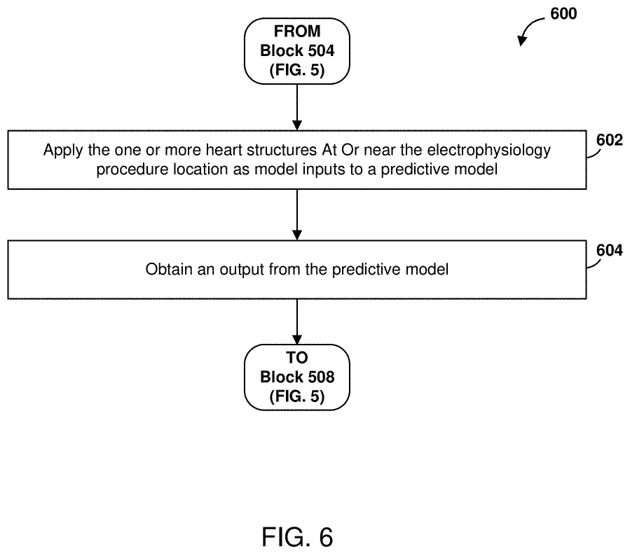

FIG. 5A is a process flow diagram illustrating an embodiment method 500 for determining a likelihood of success and/or complications from performing an electrophysiology procedure to cure arrythmia at a particular location on the heart. The operations of the method 500a may be performed by one or more processors (e.g., 404) of a diagnostic apparatus (e.g., 402).

In block 502, the diagnostic apparatus may use a patient-specific 3D cardiac activation and arrhythmia localization model to identify an area of electrophysiological interest for performing an electrophysiology procedure to treat an arrhythmia. In some embodiments, in block 502 the diagnostic apparatus may identify an area of electrophysiological interest for performing an ablation procedure. In some embodiments, in block 502 the diagnostic apparatus may identify an area of electrophysiological interest for performing a pacing procedure. In some embodiments, in block 502 the diagnostic apparatus may identify an area for conducting pace mapping for other electrophysiology therapies. In some embodiments, in block 502 the diagnostic apparatus may identify the area of electrophysiological interest as an area of earliest activation within the heart. In some embodiments, in block 502 the diagnostic apparatus may identify the area of electrophysiological interest as an area of latest activation within the heart. In some embodiments, in block 502 the diagnostic apparatus may identify the area of electrophysiological interest as an area within the heart between the earliest activation and the latest activation.

In block 504, the diagnostic apparatus may use the 3D heart model to identify heart structures near the identified area of electrophysiological interest. Non-limiting examples of structures that may be identified in block 504 may include locations of the four cardiac valves and supporting fibrous tissues, shapes, sizes and locations of the right and left atriums and right and left ventricles, locations of cardiac blood vessels and veins in the myocardium, locations of scar tissue, and other structures.

In block 506, the diagnostic apparatus may use a diagnostic prediction model to determine a prognostic indication of an electrophysiology procedure performed at the identified area of electrophysiological interest based at least in part on one or more heart structures at or near the area of electrophysiological interest. In some embodiments, the anatomical structures at or near the area of electrophysiological interest may be used as inputs to the diagnostic prediction model. In some embodiments, the diagnostic prediction model may be an AI model that was generated by training a machine learning model using information from numerous diagnostic procedures as described herein. Some embodiments may further include applying characteristics of the arrythmia to the diagnostic predictive model to obtain an output including an indication of at least one of a likelihood of success or a likelihood of complications of the electrophysiology procedure at the identified area of electrophysiological interest. In some embodiments, in block 506 the diagnostic apparatus may output to determine a prognostic indication of the likelihood that a pacing treatment at the identified area of electrophysiological interest will result in successful pacing of the patient's heart to resolve the underlying medical condition. Non-limiting examples of pacing treatments that may be assessed by the diagnostic apparatus include implanting of pacemaker leads, electrical synchronization of intra-chamber cardiac rhythms, and electrical synchronization of inter-chamber rhythms as well, such as electrical resynchronization of the right and left ventricles. In some embodiments, in block 506 the diagnostic apparatus may output to determine a prognostic indication of the likelihood that an ablation performed at the identified area of electrophysiological interest will result in successful in treatment of the patient's arrythmia condition and/or complications.

In some embodiments, the diagnostic prediction model may be stored in local memory and accessed during the operations in block 506. In other embodiments, the diagnostic prediction model may be stored in a remote server, and accessed by the diagnostic apparatus providing information regarding the electrophysiology procedure, including structures at or near the intended area of electrophysiological interest, to a remote server hosting the diagnostic prediction model, and receiving indications of the likelihood of success and/or complications from the remote server in response.

In block 508, the diagnostic apparatus may generate an output providing a prognostic indication of an electrophysiology procedure performed at the identified area of electrophysiological interest based at least in part on the likelihood of success and/or complications determined in block 506. Such an output may include a display presented on a display unit, a printout of information regarding the determined likelihood of success and/or complications provided by a printer unit, or a combination of both. The output generated in block 508 may also include electronic messages or notifications that may be transmitted by the diagnostic apparatus including indications regarding the likelihood of success and/or complications determined in block 506.

FIG. 5B is a process flow diagram illustrating an example of operations of a method 510 that may be performed to generate the patient-specific 3D cardiac activation and arrhythmia localization model that is used in block 502 of the method 500 (FIG. 5A). The operations of the method 500a may be performed by one or more processors (e.g., 404) of a diagnostic apparatus (e.g., 402).

In block 512, the diagnostic apparatus may generate a cardiac activation map including a 3D heart model that shows propagation of electrical signals through the 3D heart model based on a 3D heart model and patient ECG data recorded during arrhythmia events. The ECG measurements may be obtained using a 12-lead ECG system. In some embodiments, the ECG data may be recorded using a portable ECG recording device, such as a Holter-type device. The locations of the electrodes of the ECG device on the torso may be recorded, such as using 3D images of the patient's torso during ECG measurements as described in block 516. The positions of the electrodes in the 3D anatomical model may be used by the diagnostic apparatus for estimating the distribution, fluctuation, and/or movement of electrical activity through heart tissue. Locations on the patient of the recording leads of the ECG device may be entered in an anatomical 3D representation of the torso. The distribution, fluctuation, and/or movement of electrical activity through heart tissue used to generate the cardiac activation map may be based upon a myocardial distance function, a fastest route algorithm, shortest path algorithm, and/or fast marching algorithm. The cardiac activation map may be generated using methods as described above with reference to FIG. 3.

In block 514, the diagnostic apparatus may select a 3D reference model of the heart that includes structures of the heart, and adjust the 3D reference model based on patient DICOM image data. For example, using DICOM image data (e.g., MRI, CT, PET, ultrasound or other imagery), the diagnostic apparatus may recognize various structures within the DICOM images, determine their relative location, size and orientation, and use that information to select from a database of reference 3D anatomical models a reference 3D anatomical model that is similar to the patient's body. In some embodiments, the selected reference 3D anatomical model may include the torso of a person similar in size to the patient including a heart model with a size, orientation, and location of the heart within the torso. As part of the operations in block 514, the diagnostic apparatus may further use the structural information derived from DICOM images to adjust the location, size and orientation of corresponding structures in the 3D reference model in order to generate a patient-specific 3D anatomical model of the torso including the size, orientation, and location of the heart within the torso of the patient. Optionally, the patient-specific 3D anatomical model may also include the size, orientation and/or location of other tissues, such as the lungs and/or other organs within the torso. Optionally, scar tissue may be incorporated in the anatomical 3D representation of the heart, with the presence and location of scar tissue derived from delayed enhancement MRI images. In cases in which there is no reference 3D anatomical model similar to the patient to select, the diagnostic apparatus may generate the patient-specific 3D anatomical model based on the patient's DICOM images and 3D images of the patient's torso in block 514.

In block 516, the diagnostic apparatus may obtain a 3D image of the patient's torso. In some embodiments, this image may be obtained while ECG data is obtained from an ECG system so that the electrodes, in particular the V1-6 precordial electrodes, can be captured in the 3D image of the patient's torso. The diagnostic apparatus may receive information the positions of ECG leads relative to the anatomy of the patient from the 3D image of a patient's torso including the electrodes. Knowledge of the location of the ECG electrodes relative to the heart, and in particular the V1-6 precordial electrodes, may be especially important for accurately computing the onset location of PVC.

In some embodiments, the offsets of the electrodes from their assumed ideal locations, and in particular offsets of the V1-6 electrodes, may be determined based on a comparison of detected ECG signals of a normal heart beat to ideal ECG normal heart beat signals. For example, the offsets may be determined based on how a detected ECG signal will be affected by variations in the position of electrodes with respect to ideal electrode positions. Since the normal onset location in the SA node is known, the determined offset location may be compared to this known onset location, and the offset of the electrodes may be deduced based on the variation therebetween. As such, it may be possible to determine electrode offsets for use in generating the 3D activation map of the heart.

In block 518, the diagnostic apparatus may merge the cardiac activation map, the adjusted 3D reference model of the heart, and the 3D image of the patient's torso to form a patient-specific arrhythmia localization and cardiac activation model that includes internal structures of the heart.

In block 502 of the method 500 described with reference to FIG. 5A, the diagnostic apparatus may use the generated patient-specific arrhythmia localization and cardiac activation model to identify a location for an electrophysiology procedure and heart anatomical structures at or near the area of electrophysiological interest as described.

FIG. 6 illustrates operations 600 that may be performed in some embodiments in block 506 of the method 500 to determine indications of a likelihood of success and/or complications from performing an ablation at or near the area of electrophysiological interest.

After determining anatomical heart structures at or near an identified area of electrophysiological interest, the diagnostic apparatus may use the one or more heart structures at or near the area of electrophysiological interest as model inputs to the diagnostic predictive model in block 602. In some embodiments, this operation may be performed by applying the heart structures at or near the area of electrophysiological interest as inputs to a diagnostic predictive model stored in local memory. In some embodiments, this operation may be performed by the diagnostic apparatus sending a query to a remote server including information regarding the heart structures at or near the area of electrophysiological interest and requesting a response including indications of a likelihood of success and/or complications. As described herein, the predictive diagnostic model may be a trained AI model that accepts information regarding and intended electrophysiology procedure including information regarding heart structures at or near the area of electrophysiological interest, and provides an output indicative of a likelihood of success and/or complications from such a procedure.

In block 604, the diagnostic apparatus may obtain an output from the diagnostic predictive model. The diagnostic apparatus may then perform the operations of block 508 of the method 500 as described with reference to FIG. 5.

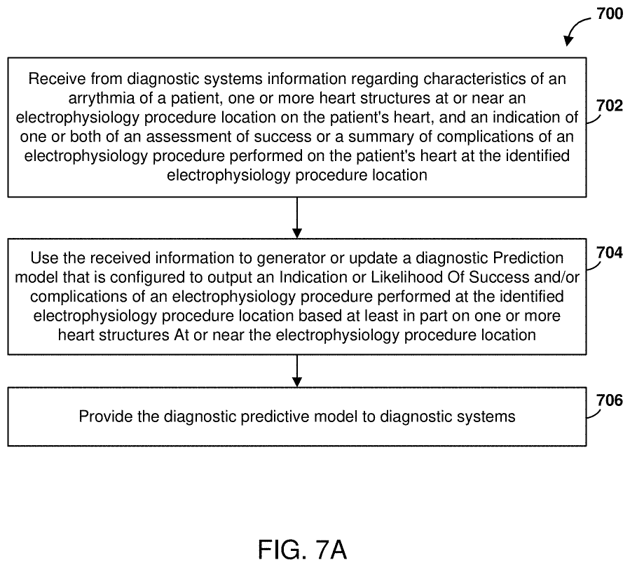

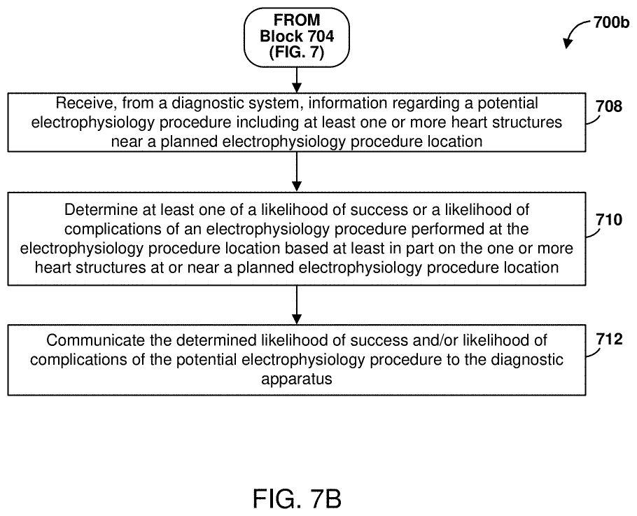

FIG. 7A is a process flow diagram illustrating an embodiment method 700 for generating a diagnostic prediction model for predicting a likelihood of success and/or complications from performing an electrophysiology procedure to cure arrythmia at a particular location on the heart based on information obtained from numerous electrophysiology procedures. The operations of the method 700 may be performed by one or more processors (e.g., 542) of a server or computing device (e.g., 418).

In block 702, the server may receive communications from multiple diagnostic systems providing information regarding characteristics of an arrhythmia of a patient, one or more heart structures at or near an area of electrophysiological interest on the patient's heart, and an indication of one or both of an assessment of success or a summary of complications of an electrophysiology procedure performed on the patient's heart at the identified area of electrophysiological interest. Such reports of electrophysiology procedure details may be received via a network, such as the Internet, automatically or through structured queries to particular diagnostic apparatuses (e.g., 402).

In block 704, the server may use the received information to generator or update a diagnostic predictive model that is configured to output an indication or likelihood of success and/or complications of an electrophysiology procedure performed at the identified area of electrophysiological interest based at least in part on one or more heart structures at or near the area of electrophysiological interest. As described herein, these operations may involve any of a variety of known analysis techniques for identifying patterns in a large set of data. In some embodiments, the operations may involve applying a database of received information about electrophysiology procedures that have been performed, including in each case identification of one or more heart structures at or near the area of electrophysiological interest and indications of success and/or complications of the electrophysiology procedure, as a training data set for training a machine learning model. Such a trained machine learning model may be configured to correlate success and/or complications of electrophysiology procedures to structures at or near the area of electrophysiological interest in a manner that enables receiving information regarding heart structures at or near a planned area of electrophysiological interest and outputting a probability or likelihood of success or complications of performing such an electrophysiology procedure. The operations in block 704 may be performed on a continuous basis as new information regarding performed electrophysiology procedures is received, enabling the diagnostic predictive model to be refined over time as more information about electrophysiology procedure successes and complications is received.

In block 706, the server may provide the diagnostic predictive model to diagnostic apparatuses. In some embodiments, the server may download the diagnostic predictive model to diagnostic apparatuses via a network (e.g., the Internet), such as via a registration and configuration procedure performed by diagnostic apparatuses. In some embodiments, the server may periodically or episodically download updates or refinements to the diagnostic predictive model to diagnostic apparatuses.

FIG. 7B is a process flow diagram illustrating example operations that may be performed by a server to provide diagnostic apparatuses with determinations of likelihood of success or likelihood of complications for potential electrophysiology procedures according to some embodiments. The operations of the method 700 may be performed by one or more processors (e.g., 542) of a server or computing device (e.g., 418).

After the server has generated or refined the diagnostic predictive model in block 704 of the method 700 (FIG. 7A), the server may receive communications from a diagnostic system in block 708, including information regarding a potential electrophysiology procedure and information regarding at least one or more heart structures at or near a planned area of electrophysiological interest. The communications from the diagnostic apparatus may be received in the form of a request for service or query, which may be received via a network (e.g., the Internet). In addition to information regarding heart structures at or near the planned area of electrophysiological interest, communications from the diagnostic system may include further information, such as the type of arrhythmia being experienced by the patient, medical history information, and other information that may be pertinent to assessing the likelihood of success and/or complications for performing the planned electrophysiology procedure.

In block 710, the server may determine a prognostic indication of an electrophysiology procedure performed at the identified area of electrophysiological interest based at least in part on one or more heart structures near the area of electrophysiological interest. In some embodiments, the prognostic indication may include at least one of a likelihood of success or a likelihood of complications of the electrophysiology procedure performed at the area of electrophysiological interest based at least in part on the one or more's heart structures at or near the planned area of electrophysiological interest. As described herein, these operations may involve using the identified one or more heart structures at or near the planned area of electrophysiological interest as inputs to the diagnostic predictive model (i.e., the model developed in method 700) and receiving an output from that model indicating a likelihood of success and/or a likelihood of complications.

In block 712, the server may communicate an indication of the determined likelihood of success and/or likelihood of complications of the potential electrophysiology procedure to the diagnostic apparatus, such as via a network (e.g., the Internet). In these operations, the server may format information regarding the determined likelihood of success and/or complications in a data structure that can be used by the diagnostic apparatus to generate an output for a physician, such as in block 508 of the method 500 (FIG. 5).

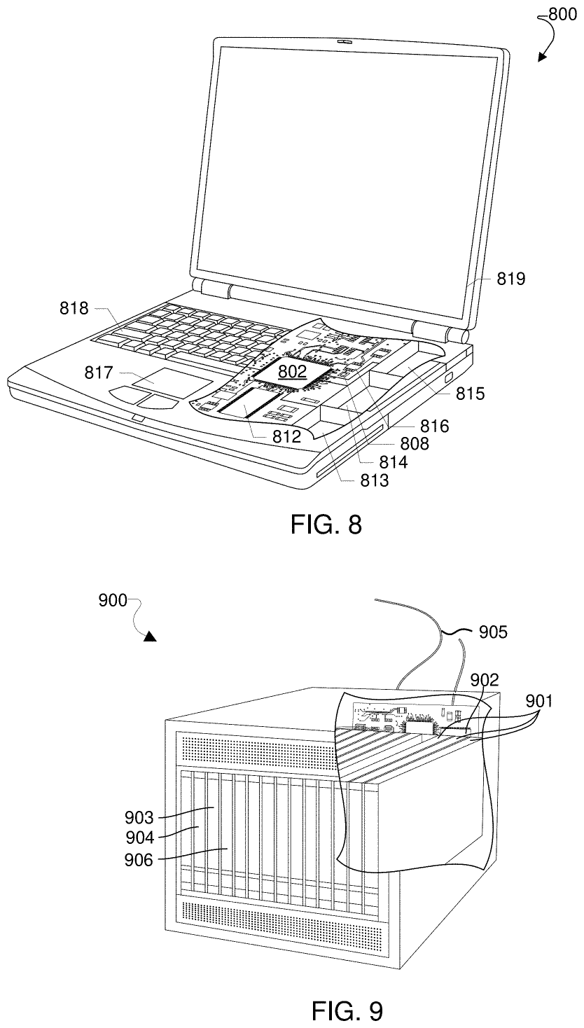

The various embodiments (including, but not limited to, embodiments described above with reference to FIGS. 1-7) may be implemented in a wide variety of computing systems include a laptop computer 800, an example of which is illustrated in FIG. 8. Many laptop computers include a touchpad touch surface 817 that serves as the computer's pointing device. A laptop computer 800 will typically include a processor 802 coupled to volatile memory 812 and a large capacity nonvolatile memory, such as a disk drive 813 of FLASH memory. Additionally, the computer 800 may have one or more antenna 808 for sending and receiving electromagnetic radiation that may be connected to a wireless data link (e.g., Bluetooth or Wi-Fi) and/or cellular telephone transceiver 816 coupled to the processor 802. The computer 800 may also include a floppy disc drive 814 and a compact disc (CD) drive 815 coupled to the processor 802. In a notebook configuration, the computer housing includes the touchpad 817, the keyboard 818, and the display 819 all coupled to the processor 802. Other configurations of the computing device may include a computer mouse or trackball coupled to the processor (e.g., via a USB input) as are well known, which may also be used in conjunction with the various embodiments.

The various embodiments (including, but not limited to, embodiments described above with reference to FIGS. 1-7) may also be implemented in fixed computing systems, such as any of a variety of commercially available servers. An example server 900 is illustrated in FIG. 9. Such a server 900 typically includes one or more multicore processor assemblies 901 coupled to volatile memory 902 and a large capacity nonvolatile memory, such as a disk drive 904. As illustrated in FIG. 9, multicore processor assemblies 901 may be added to the server 900 by inserting them into the racks of the assembly. The server 900 may also include a floppy disc drive, compact disc (CD) or digital versatile disc (DVD) disc drive 906 coupled to the processor 901. The server 900 may also include network access ports 903 coupled to the multicore processor assemblies 901 for establishing network interface connections with a network 905, such as a local area network coupled to other broadcast system computers and servers, the Internet, the public switched telephone network, and/or a cellular data network.

The foregoing embodiment descriptions and the process flow diagrams are provided merely as illustrative examples and are not intended to require or imply that the steps of the various embodiments must be performed in the order presented. By way of example but not limitation, the scope of the claims is intended to include a site of electrophysiological interest, not just an identified ablation site, and that this site could include, but not be limited to the identified earliest activation or the latest activation. The scope of the claims is intended to cover all cardiac electrophysiology procedures, not simply cardiac ablation, including pace mapping for other electrophysiology therapies, implanting of pacemaker leads, electrical synchronization of intra-chamber cardiac rhythms, and electrical synchronization of inter-chamber rhythms as well, such as electrical resynchronization of the right and left ventricles.

As will be appreciated by one of skill in the art the order of steps in the foregoing embodiment methods may be performed in any order. For example, the sequence in which DICOM images, ECG data and 3D images of the patient are obtained may be different from the order presented in the figures and described above.

Words such as “thereafter,” “then,” “next,” etc. are not intended to limit the order of the steps; these words are simply used to guide the reader through the description of the methods. Further, any reference to claim elements in the singular, for example, using the articles “a,” “an” or “the” is not to be construed as limiting the element to the singular.

The various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, operations and modules have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the claims.

The hardware used to implement the various illustrative operations and modules disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but, in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration. Alternatively, some steps or methods may be performed by circuitry that is specific to a given function.

In one or more exemplary aspects, the functions described may be implemented in hardware, software, firmware, or any combination thereof. If implemented in software, the functions may be stored as one or more processor-executable instructions or code on a non-transitory computer-readable medium or non-transitory processor-readable medium. The operations of a method or algorithm disclosed herein may be embodied in a processor-executable software module and/or processor-executable instructions, which may reside on a non-transitory computer-readable or non-transitory processor-readable storage medium. Non-transitory server-readable, computer-readable or processor-readable storage media may be any storage media that may be accessed by a computer or a processor. By way of example but not limitation, such non-transitory server-readable, computer-readable or processor-readable media may include RAM, ROM, EEPROM, FLASH memory, CD-ROM or other optical disk storage, magnetic disk storage or other magnetic storage devices, or any other medium that may be used to store desired program code in the form of instructions or data structures and that may be accessed by a processor or computer. Disk and disc, as used herein, includes compact disc (CD), laser disc, optical disc, digital versatile disc (DVD), floppy disk, and Blu-ray disc where disks usually reproduce data magnetically, while discs reproduce data optically with lasers. Combinations of the above are also included within the scope of non-transitory server-readable, computer-readable and processor-readable media. Additionally, the operations of a method or algorithm may reside as one or any combination or set of codes and/or processor-executable instructions on a non-transitory server-readable, processor-readable medium and/or computer-readable medium, which may be incorporated into a computer program product.

The preceding description of the disclosed embodiments is provided to enable any person skilled in the art to make or use the present invention. Various modifications to these embodiments will be readily apparent to those skilled in the art, and the generic principles defined herein may be applied to other embodiments without departing from the scope of the claims. Thus, the claims are not intended to be limited to the embodiments shown herein but are to be accorded the widest scope consistent with the following claims and the principles and novel features disclosed herein.

What is claimed is:

1. A method performed by a diagnostic apparatus for determining a likely outcome of an electrophysiology procedure for treating a heart arrythmia in a patient, comprising:
using a patient-specific three-dimensional (3D) cardiac activation and arrythmia localization model to identify an electrophysiological area of interest for performing an electrophysiology procedure to treat the arrythmia;
using a patient-specific 3D heart model to identify heart structures near the identified area of electrophysiological interest;
determining a prognostic indication of an electrophysiology procedure performed at the identified area of electrophysiological interest based at least in part on one or more heart structures near the area of electrophysiological interest; and
generating an output providing a prognostic indication of an electrophysiology procedure at the identified area of electrophysiological interest based at least in part on the determined likelihood of success.

2. The method of claim 1, wherein the area of electrophysiological interest comprises an area of earliest activation within the heart.

3. The method of claim 1, wherein the area of electrophysiological interest comprises an area of latest activation within the heart.

4. The method of claim 1, wherein the area of electrophysiological interest comprises an area within the heart between an area of earliest activation and an area of latest activation.

5. The method of claim 1, further comprising generating the patient-specific 3D cardiac activation and arrythmia localization model by:
generating a cardiac activation map comprising a 3D heart model that shows propagation of electrical signals through the 3D heart model based on patient electrocardiogram (ECG) data recording during arrythmia events and a 3D heart model;
selecting a 3D reference model of the heart that includes structures of the heart and adjusting the 3D reference model of the heart based on patient Digital Imaging and Communications in Medicine (DICOM) image data;
obtaining a 3D image of the patient's torso; and
merging the cardiac activation map, the adjusted 3D reference model of the heart, and the 3D image of the patient's torso to form a patient-specific arrythmia localization and cardiac activation model that includes internal structures of the heart.

6. The method of claim 1, wherein determining the prognostic indication of the electrophysiology procedure comprises determining at least one of a likelihood of success or a likelihood of complications of the electrophysiology procedure at the identified area of electrophysiological interest.

7. The method of claim 6, wherein determining at least one of a likelihood of success or a likelihood of complications comprises:
applying the one or more heart structures near the area of electrophysiological interest as model inputs to a predictive model; and
obtaining an output from the diagnostic predictive model.

8. The method of claim 7, further comprising applying characteristics of the arrythmia to the diagnostic predictive model.

9. The method of claim 7, wherein:
the diagnostic predictive model is maintained in a remote server;
applying the one or more heart structures near the area of electrophysiological interest as model inputs to the diagnostic predictive model comprises uploading the one or more heart structures near the area of electrophysiological interest to the remote server; and
obtaining an output from the diagnostic predictive model comprises receiving the output from the remote server.

10. The method of claim 7, wherein the diagnostic predictive model is stored in memory of the diagnostic apparatus.

11. The method of claim 10, further comprising downloading the diagnostic predictive model or an update to the diagnostic predictive model to memory from a remote server.

12. The method of claim 1, further comprising uploading to a remote server information regarding characteristics of the arrythmia, information regarding the one or more heart structures near the area of electrophysiological interest, and an indication of one or both of an assessment of success or a summary of complications of a performed electrophysiology procedure performed at the identified area of electrophysiological interest.

13. The method of claim 1, wherein the electrophysiology procedure comprises an ablation procedure.

14. The method of claim 1, wherein the electrophysiology procedure comprises a pacing procedure.

15. A diagnostic system, comprising:
a memory; and
a processor coupled to the memory and configured with processor-executable instructions to perform operations comprising:
generating a patient-specific three-dimensional (3D) cardiac activation and arrythmia localization model identifying an area of electrophysiological interest for performing an electrophysiology procedure to treat the arrythmia;
using a patient-specific 3D heart model to identify heart structures near the identified area of electrophysiological interest;
determining a prognostic indication of an electrophysiology procedure performed at the identified area of electrophysiological interest based at least in part on one or more heart structures near the area of electrophysiological interest; and
generating an output providing a prognostic indication of an electrophysiology procedure at the identified area of electrophysiological interest based at least in part on the determined prognostic indication.

16. The diagnostic system of claim 15, wherein the area of electrophysiological interest comprises an area of earliest activation within the heart.

17. The diagnostic system of claim 15, wherein the area of electrophysiological interest comprises an area of latest activation within the heart.

18. The diagnostic system of claim 15, wherein the area of electrophysiological interest comprises an area within the heart between an area of earliest activation and an area of latest activation.

19. The diagnostic system of claim 15, wherein the processor is further configured with processor-executable instructions to perform operations such that generating a patient-specific 3D cardiac activation and arrythmia localization model identifying an area of electrophysiological interest for performing an electrophysiology procedure to treat the arrythmia comprises:
generating a cardiac activation map comprising a 3D heart model that shows propagation of electrical signals through the 3D heart model based on patient electrocardiogram (ECG) data recording during arrythmia events and a 3D heart model;
selecting a 3D reference model of the heart that includes structures of the heart and adjusting the 3D reference model of the heart based on patient Digital Imaging and Communications in Medicine (DICOM) image data;
obtaining a 3D image of the patient's torso;
merging the cardiac activation map, the adjusted 3D reference model of the heart, and the 3D image of the patient's torso to form a patient-specific arrythmia localization and cardiac activation model that includes internal structures of the heart; and
using the patient-specific arrythmia localization and cardiac activation model to identify an area of electrophysiological interest for performing an electrophysiology procedure to treat the arrythmia.

20. The diagnostic system of claim 19, wherein the processor is further configured with processor-executable instructions to perform operations such that determining the prognostic indication of the electrophysiology procedure comprises determining at least one of a likelihood of success or a likelihood of complications of the electrophysiology procedure at the identified area of electrophysiological interest.

21. The diagnostic system of claim 19, wherein the processor is further configured with processor-executable instructions to perform operations such that determining at least one of a likelihood of success or a likelihood of complications comprises:
applying the one or more heart structures near the area of electrophysiological interest as model inputs to a predictive model; and
obtaining an output from the diagnostic predictive model.

22. The diagnostic system of claim 21, wherein the processor is further configured with processor-executable instructions to perform operations further comprising applying characteristics of the arrythmia to the diagnostic predictive model.

23. The diagnostic system of claim 21, wherein the processor is further configured with processor-executable instructions to perform operations such that:
the diagnostic predictive model is maintained in a remote server;
applying the one or more heart structures near the area of electrophysiological interest as model inputs to the diagnostic predictive model comprises receiving information regarding the one or more heart structures near the area of electrophysiological interest from a diagnostic system and applying the received information regarding the one or more heart structures near the area of electrophysiological interest as inputs to the diagnostic predictive model; and
obtaining an output from the diagnostic predictive model comprises transmitting the output from the diagnostic predictive model to the diagnostic system.

24. The diagnostic system of claim 21, wherein the processor is further configured with processor-executable instructions to perform operations such that the diagnostic predictive model is stored in memory coupled to the processor.

25. The diagnostic system of claim 21, wherein the processor is further configured with processor-executable instructions to perform operations further comprising downloading the diagnostic predictive model or an update to the diagnostic predictive model to memory from a remote server.

26. The diagnostic system of claim 15, wherein the processor is further configured with processor-executable instructions to perform operations further comprising uploading to a remote server information regarding characteristics of the arrythmia, information regarding the one or more heart structures near the area of electrophysiological interest, and an indication of one or both of an assessment of success or a summary of complications of a performed electrophysiology procedure performed at the identified area of electrophysiological interest.

27. The diagnostic system of claim 15, wherein the electrophysiology procedure comprises an ablation procedure.

28. The diagnostic system of claim 15, wherein the electrophysiology procedure comprises a pacing procedure.

29. A method performed by a server, comprising:
receiving, from diagnostic systems, information regarding characteristics of an arrythmia of a patient, one or more heart structures near an area of electrophysiological interest on the patient's heart, and an indication of one or both of an assessment of success or a summary of complications of an electrophysiology procedure performed on the patient's heart at the identified area of electrophysiological interest;
using the received information to generator or update a diagnostic model that is configured to output a prognostic indication of an electrophysiology procedure performed at the identified area of electrophysiological interest based at least in part on one or more heart structures near the area of electrophysiological interest; and
providing the diagnostic predictive model to diagnostic systems.

30. The method of claim 29, wherein the diagnostic model is configured to output the prognostic indication of an electrophysiology procedure including at least one of a likelihood of success or a likelihood of complications of an electrophysiology procedure performed at the identified area of electrophysiological interest based at least in part on one or more heart structures near the area of electrophysiological interest.

31. The method of claim 29, wherein providing the diagnostic predictive model to diagnostic systems comprises:
receiving, from a diagnostic system, information regarding a potential electrophysiology procedure including information regarding at least one or more heart structures near an area of electrophysiological interest;
determining at least one of a likelihood of success or a likelihood of complications of an electrophysiology procedure at the area of electrophysiological interest based at least in part on the one or more heart structures near the area of electrophysiological interest; and
communicating the determined likelihood of success or likelihood of complications of the potential electrophysiology procedure to the diagnostic system.
